(19) United States
(12) Patent Application Publication
(10) Pub. No.: US 2026/0294509 A1
(43) Pub. Date: Oct. 1, 2026

(54) RF SEPTAL MYECTOMY TOOL

(71) Applicant: RAMBAM MEDTECH LTD., Haifa (IL)

(72) Inventors: Yair FELD, Haifa (IL); Manhal HABIB, Haifa (IL)

(73) Assignee: RAMBAM MEDTECH LTD., Haifa (IL)

(21) Appl. No.: 19/480,871

(22) PCT Filed: May 7, 2024
(86) PCT No.: PCT/IL2024/050431

§ 371 Date: Nov. 3, 2025

Related U.S. Application Data
(60) Provisional application No. 63/465,074, filed on May 9, 2023.

(51) Int. Cl. A61B 18/12; A61B 5/33; A61B 5/361; A61B 17/00; A61B 17/32; A61B 18/00; A61B 18/14; A61B 90/00; A61N 1/39

(52) U.S. Cl. CPC A61B 18/1233; A61B 5/33; A61B 5/361; A61B 18/1492; A61B 2017/00247; A61B 2017/320064; A61B 2018/00601; A61B 2018/1407; A61B 2018/1475; A61B 2090/0427; A61N 1/3987

(57) ABSTRACT

A percutaneous electrosurgical system (20) is provided for use with an electrosurgical radiofrequency (RF) generator unit (22), for applying RF energy to cardiac tissue within a cardiac chamber of a heart. The electrosurgical system (20) includes a transcatheter electrosurgical device (30), which includes (a) a catheter (32) and (b) a distal tip (34) disposable at a distal end of the catheter (32) and including an active electrode (36). Electrical wiring (40) is configured to electrically couple the electrosurgical RF generator unit (22) to the active electrode (36) and a dispersive electrode (38), so as to provide RF energy from the electrosurgical RF generator unit (22) to the active electrode (36). A control unit (42) is configured to perform cardiac sensing for ventricular fibrillation that may be induced by the RF energy, and upon sensing the ventricular fibrillation, apply defibrillation current to the heart. Other embodiments are also described.

### CROSS-REFERENCE TO RELATED APPLICATIONS

The present application claims priority from U.S. Provisional Application 63/465,074, filed May 9, 2023, which is assigned to the assignee of the present application and incorporated herein by reference.

### FIELD OF INVENTION

Some applications of the present invention relate in general to septal myectomy. More specifically, some applications of the present invention relate to applying RF energy to cardiac tissue in order to cut it.

### BACKGROUND

Septal myectomy, a treatment for hypertrophic cardiomyopathy, is performed as an open-heart surgical procedure in which a portion of the septum is removed in order to prevent the obstruction of blood flow from the left ventricle to the aorta.

### SUMMARY OF THE INVENTION

In accordance with an application of the present invention, a transcatheter electrosurgical device cuts excess cardiac tissue, e.g., septal myocardial tissue. In some applications, cutting is performed with a cutting ring supplied with radiofrequency (RF) energy. Pulling of the cutting ring while the cutting ring contacts the septal myocardial tissue applies the RF energy to the tissue, thereby cutting the septal myocardial tissue and trapping the cut septal myocardial tissue in a collection chamber of the transcatheter electrosurgical device.

In accordance with an application of the present invention, the transcatheter electrosurgical device applies a defibrillation current to the heart upon sensing ventricular fibrillation that may be induced by the RF energy.

There is therefore provided, in accordance with some applications of the present invention, a percutaneous electrosurgical system for use with an electrosurgical radiofrequency (RF) generator unit, for applying RF energy to cardiac tissue within a cardiac chamber of a heart, the electrosurgical system including:
a transcatheter electrosurgical device, the transcatheter electrosurgical device including (a) a catheter and (b) a distal tip, the distal tip disposable at a distal end of the catheter and including an active electrode;
a dispersive electrode;
electrical wiring, which is configured to electrically couple the electrosurgical RF generator unit to the active electrode and the dispersive electrode, so as to provide RF energy from the electrosurgical RF generator unit to the active electrode; and
a control unit, which includes circuitry and is configured to:
   perform cardiac sensing for ventricular fibrillation that may be induced by the RF energy, and
   upon sensing the ventricular fibrillation, apply defibrillation current to the heart.

In an application, the control unit is configured to disconnect the active electrode from the electrosurgical RF generator unit upon sensing the ventricular fibrillation.

In an application, the distal tip further includes a defibrillation electrode, and the control unit is configured to apply the defibrillation current to the heart via the defibrillation electrode.

In an application, the control unit is configured to apply the defibrillation current to the heart via the active electrode.

In an application, the distal tip further includes a defibrillation electrode, and the control unit is configured to perform the cardiac sensing using the defibrillation electrode. In an application, the control unit is configured to perform the cardiac sensing using the active electrode.

In an application, the distal tip of the transcatheter electrosurgical device includes a collection chamber, and the distal tip further includes insulating material disposed between the active electrode and the collection chamber.

In an application, the electrosurgical system is configured to perform transcatheter septal myectomy on the heart.

In an application:
the distal tip of the transcatheter electrosurgical device includes a collection chamber,
the active electrode is shaped as an electrically-conductive cutting ring, and
the distal tip is arranged such that motion of the cutting ring, while the cutting ring contacts septal myocardial tissue of the heart and is provided with the RF energy from the RF generator unit, cuts the septal myocardial tissue and traps the cut septal myocardial tissue in the collection chamber.

In an application:
the collection chamber has an open proximal end and a closed distal end,
the cutting ring is coupled to the open proximal end of the collection chamber,
the transcatheter electrosurgical device further includes an elongate cutting-ring puller, which is coupled to the cutting ring, and
the distal tip is arranged such that pulling of the elongate cutting-ring puller in a proximal direction, while the cutting ring contacts the septal myocardial tissue and is provided with RF energy from the RF generator unit, pulls the cutting ring and the open proximal end of the collection chamber in the proximal direction, thereby cutting the septal myocardial tissue and trapping the cut septal myocardial tissue in the collection chamber.

There is further provided, in accordance with an application of the present invention, an electrosurgical system for use with an electrosurgical radiofrequency (RF) generator unit, for performing percutaneous transcatheter septal myectomy on a heart, the electrosurgical system including:
(i) a transcatheter electrosurgical device, including:
   (a) a catheter;
   (b) a distal tip, which is disposable at a distal end of the catheter, and which includes (1) a collection chamber having an open proximal end and a closed distal end; and (2) an electrically-conductive cutting ring, which is coupled to the open proximal end of the collection chamber; and
   (c) an elongate cutting-ring puller, which is coupled to the cutting ring;
(ii) a dispersive electrode; and
(iii) electrical wiring, which is configured to electrically couple the electrosurgical RF generator unit to the cutting ring and the dispersive electrode, and so as to provide RF energy from the electrosurgical RF generator unit to the cutting ring,
the distal tip is arranged such that pulling of the elongate cutting-ring puller in a proximal direction, while the cutting ring contacts septal myocardial tissue of the heart and is provided with the RF energy from the RF generator unit, pulls the cutting ring and the open proximal end of the collection chamber in the proximal direction, thereby cutting the septal myocardial tissue and trapping the cut septal myocardial tissue in the collection chamber.

In an application:
the collection chamber is elongatable from a short state to an elongated state, and
the distal tip is arranged such that the pulling of the elongate cutting-ring puller in the proximal direction pulls the cutting ring and the open proximal end of the collection chamber in the proximal direction, thereby cutting the septal myocardial tissue, elongating the collection chamber in the proximal direction, and trapping the cut septal myocardial tissue in the collection chamber as the collection chamber elongates.

In an application, the collection chamber is accordion-pleated.

In an application:
the elongate cutting-ring puller is shaped so as to define a puller lumen, and
the transcatheter electrosurgical device further includes an elongate guide, which is slidably disposed partially within the puller lumen, and which is arranged to guide the elongate cutting-ring puller in the proximal direction during the pulling of the elongate cutting-ring puller.

In an application, the electrosurgical system is for use with a guidewire, and the elongate guide is shaped so as to define a guide lumen, which has a distal end opening distal to the closed distal end of the collection chamber, so as to allow passage of the guidewire distally beyond the collection chamber.

In an application, the transcatheter electrosurgical device further includes a distal atraumatic tip, which is (a) disposed distal to the collection chamber, (b) coupled to a distal portion of the elongate guide, and (c) shaped so as to allow passage of the guidewire from the guide lumen to distally beyond the tip.

In an application:
the distal tip further includes an atraumatic valve protector, which is disposed proximal to the cutting ring, and
the distal tip is arranged such that pulling of the elongate cutting-ring puller in the proximal direction pulls the cutting ring toward the atraumatic valve protector.

In an application, the atraumatic valve protector is shaped so as to define an opening, and the elongate cutting-ring puller is slidably disposed passing through the opening.

The present invention will be more fully understood from the following detailed description of applications thereof, taken together with the drawings, in which:

### BRIEF DESCRIPTION OF THE DRAWINGS

FIG. 1 is a schematic illustration of a percutaneous electrosurgical system for use with an electrosurgical radiofrequency (RF) generator unit, for applying RF energy to cardiac tissue within a cardiac chamber of a heart, in accordance with some applications of the present invention;

FIGS. 2A-C are schematic illustrations that show a transcatheter electrosurgical device, in accordance with some applications of the present invention;

FIG. 3 is a schematic illustration of electrical wiring, which is configured to electrically couple the electrosurgical RF generator unit to the active electrode and the dispersive electrode, so as to provide RF energy from the electrosurgical RF generator unit to the active electrode, in accordance with some applications of the present invention;

FIG. 4 is a schematic illustration of the circuitry of a control unit, in accordance with some applications of the present invention; and

FIGS. 5A-D are schematic illustrations that show the cutting of septal myocardial tissue, in accordance with some applications of the present invention.

### DETAILED DESCRIPTION OF APPLICATIONS OF THE INVENTION

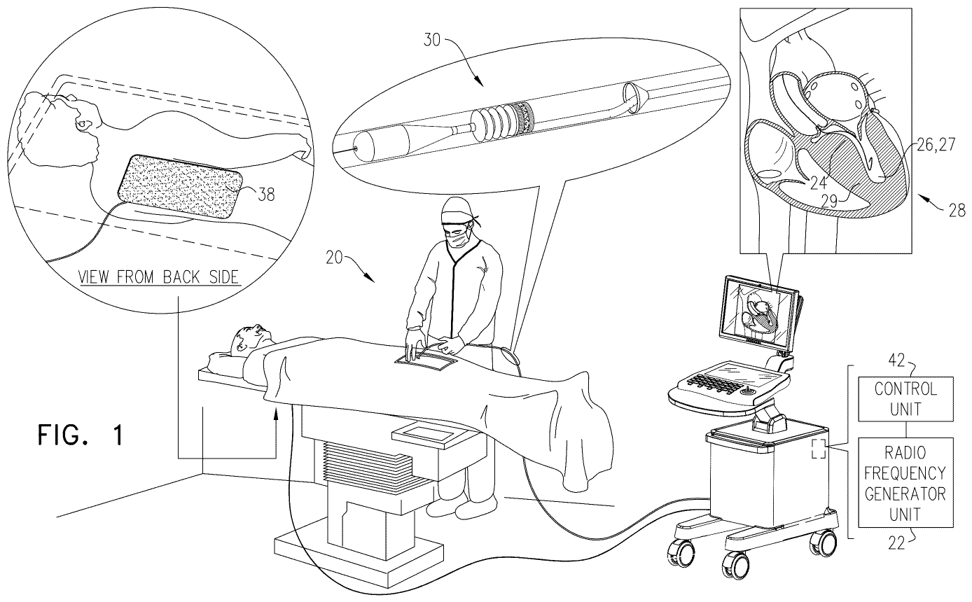

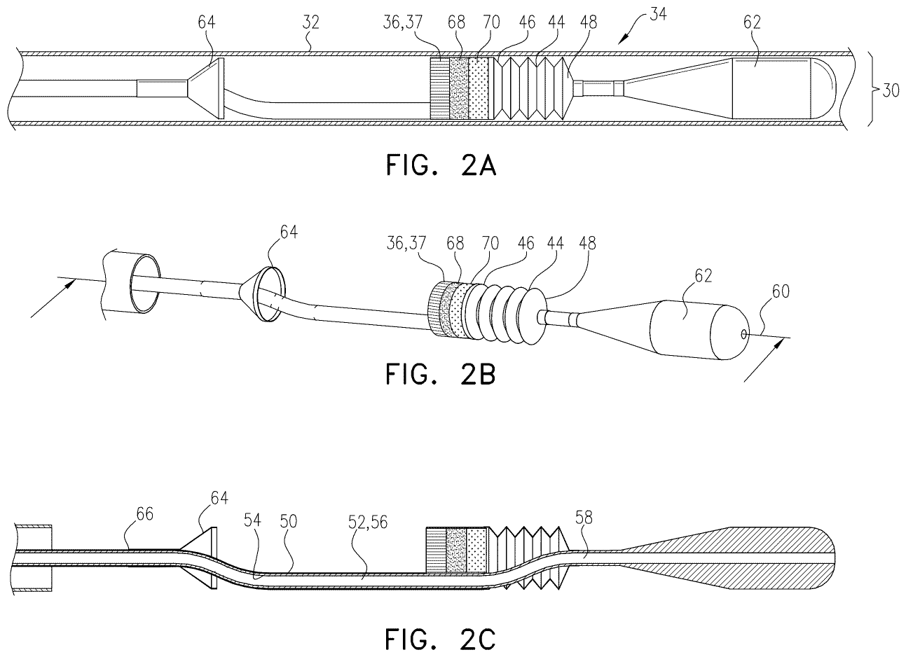

Reference is now made to FIGS. 1 and 2A-C, which are schematic illustrations of a percutaneous electrosurgical system 20 for use with an electrosurgical RF generator unit 22, in accordance with some applications of the present invention. RF generator unit 22 treats a patient by applying RF energy to cardiac tissue 24 within a cardiac chamber 26 of the patient's heart 28. Typically, cardiac chamber 26 is the patient's left ventricle 27, and electrosurgical system 20 is configured to perform transcatheter septal myectomy on an interventricular septum 29 of heart 28. Septal myectomy as described herein may be used, for example, to relieve left ventricular outflow tract obstruction, such as in patients with hypertrophic cardiomyopathy, and/or in preparation for another treatment (e.g., transcatheter prosthetic mitral valve implantation).

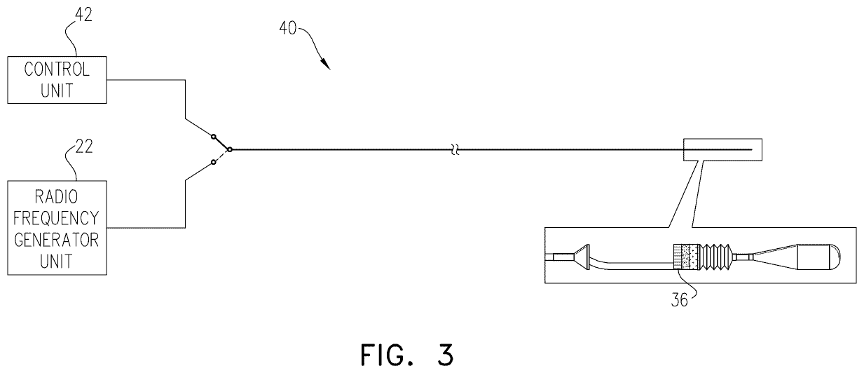

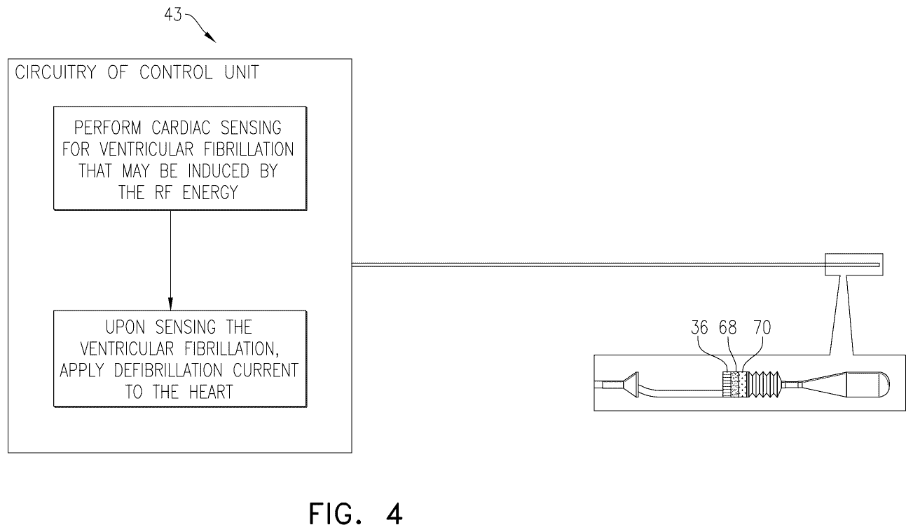

Typically, electrosurgical system 20 comprises a transcatheter electrosurgical device 30, which comprises a catheter 32 (FIG. 2A) and a distal tip 34 disposable at the distal end of the catheter. Distal tip 34 comprises an active electrode 36 of electrosurgical system 20. Active electrode 36 is typically shaped as an electrically-conductive cutting ring 37 (FIG. 2A). Electrosurgical system 20 further comprises a dispersive electrode 38 (FIG. 1), electrical wiring 40 (FIG. 3), and a control unit 42.

Typically, distal tip 34 of transcatheter electrosurgical device 30 also comprises a collection chamber 44. The collection chamber is expandable (for example, by being accordion-pleated) from a short state to an elongated state. Collection chamber 44 typically has an open proximal end 46 and a closed distal end 48, and cutting ring 37 is coupled to open proximal end 46 of collection chamber 44. The transcatheter electrosurgical device further comprises an elongate cutting-ring puller 50 (FIG. 2C), which is coupled to cutting ring 37 and can be used by the physician to pull cutting ring 37 proximally.

For some applications, elongate cutting-ring puller 50 is a tube having a puller lumen 52, which is slidably disposed over an elongate guide 54 of transcatheter electrosurgical device 30. Elongate guide 54 is arranged to guide elongate cutting-ring puller 50 in the proximal direction during the pulling of elongate cutting-ring puller 50. Typically, a handle (not shown) of transcatheter electrosurgical device 30, or a physician, holds elongate guide 54 steady while pulling the elongate cutting-ring puller 50 in order to cut excess cardiac tissue 24.

Typically, elongate guide 54 is shaped so as to define a guide lumen 56, which has a distal end opening 58 distal to closed distal end of collection chamber 44, so as to allow passage of a guidewire 60 distally beyond the collection chamber and through a distal atraumatic tip 62 of transcatheter electrosurgical device 30 (FIG. 2B). Distal atraumatic tip 62 is disposed distal to collection chamber 44, and may also be coupled to a distal portion of elongate guide 54. During the insertion of transcatheter electrosurgical device 30, the device is passed over guidewire 60 into the patient's heart.

Typically, distal tip 34 further comprises an atraumatic valve protector 64, which is disposed proximal to cutting ring 37, and pulling of elongate cutting-ring puller 50 pulls cutting ring 37 toward atraumatic valve protector 64. Typically, elongate cutting-ring puller 50 is slidably disposed passing through an opening 66 of atraumatic valve protector 64 (FIG. 2C).

Reference is now made to FIG. 3, which depicts electrical wiring 40, in accordance with some applications of the present invention. Wiring 40 is typically configured to electrically couple the electrosurgical RF generator unit 22 to active electrode 36 and dispersive electrode 38, thereby providing RF energy from electrosurgical RF generator unit 22 to active electrode 36.

Reference is now made to FIG. 4, which depicts circuitry 43 of control unit 42, in accordance with some application of the present invention. Circuitry 43 typically perform cardiac sensing for ventricular fibrillation that may be induced by the RF energy. Upon sensing the ventricular fibrillation, circuitry 43 may be activated to apply defibrillation current to heart 28.

For some applications, control unit 42 terminates the operation of electrosurgical RF generator unit 22 or disconnects active electrode 36 from electrosurgical RF generator unit 22 upon sensing ventricular fibrillation (FIG. 3). In an application, distal tip 34 further comprises a defibrillation electrode 68 (FIG. 2A), and control unit 42 applies the defibrillation current to the heart via defibrillation electrode 68. Alternatively, control unit 42 applies the defibrillation current to the heart via active electrode 36.

In an application, control unit 42 uses active electrode 36 (or, alternatively, defibrillation electrode 68) to perform the cardiac sensing.

Typically, an insulating material 70 (FIG. 2A) is disposed between the electrodes and collection chamber 44. Insulating material 70 provides collection chamber 44 protection against damage due to RF energy.

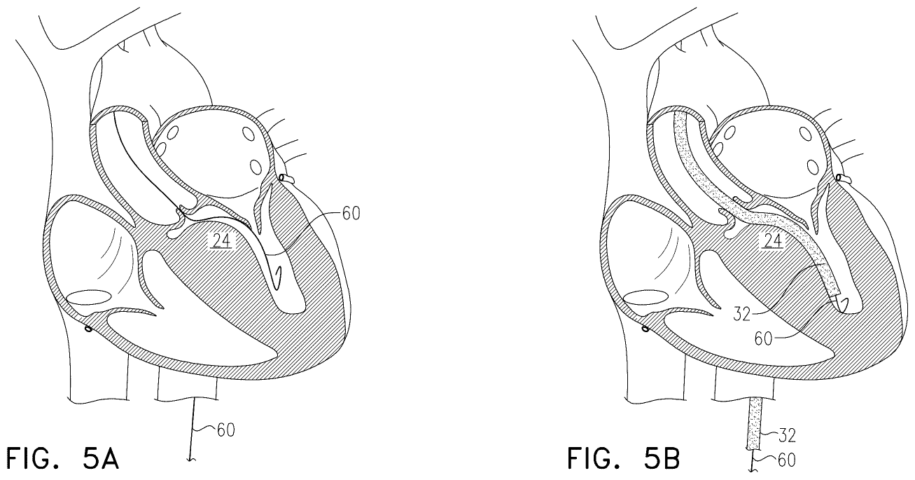

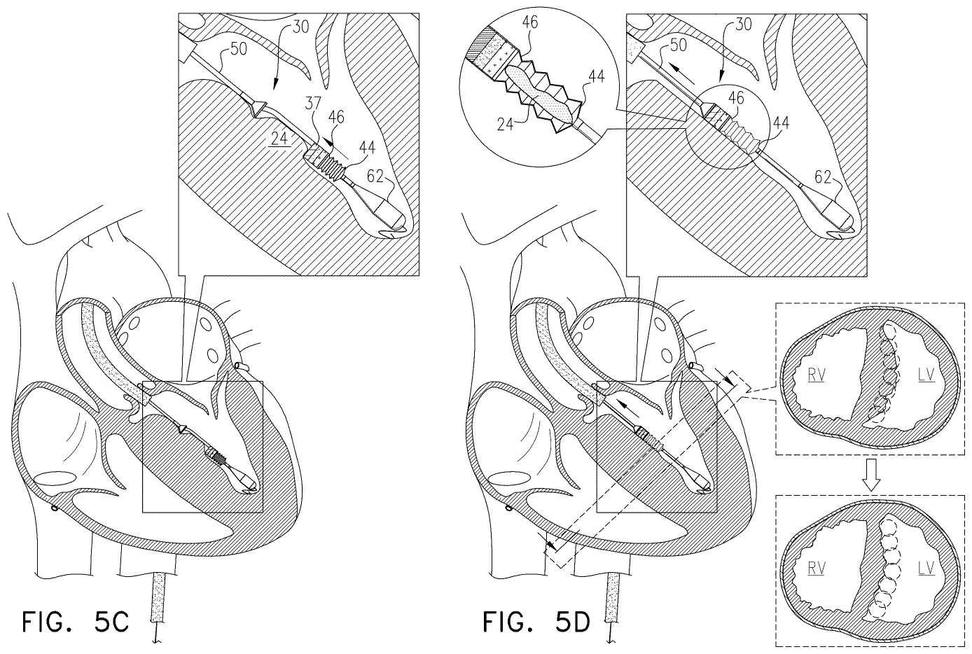

Reference is now made to FIGS. 5A-D, which show the cutting of septal cardiac tissue 24, in accordance with some applications of the present invention. Typically, elongate cutting-ring puller 50 is pulled by the physician in a proximal direction (FIGS. 5C-D), while cutting ring 37 contacts the septal cardiac tissue 24 and is provided with RF energy from RF generator unit 22. This proximal motion of cutting ring 37 pulls open proximal end 46 of collection chamber 44 in the proximal direction, thereby cutting septal cardiac tissue 24 and elongating the collection chamber 44 in the proximal direction. The cut septal myocardial tissue is captured in collection chamber 44 as collection chamber 44 elongates. This cut septal tissue is removed from the patient's body, and, if desired, the procedure can be repeated one or more times (FIG. 5D).

It will be appreciated by persons skilled in the art that the present invention is not limited to what has been particularly shown and described hereinabove. Rather, the scope of the present invention includes both combinations and subcombinations of the various features described hereinabove, as well as variations and modifications thereof that are not in the prior art, which would occur to persons skilled in the art upon reading the foregoing description.

1. A percutaneous electrosurgical system for use with an electrosurgical radiofrequency (RF) generator unit, for applying RF energy to cardiac tissue within a cardiac chamber of a heart, the electrosurgical system comprising:
a transcatheter electrosurgical device, comprising (a) a catheter and (b) a distal tip, the distal tip disposable at a distal end of the catheter and comprising an active electrode;
a dispersive electrode;
electrical wiring, which is configured to electrically couple the electrosurgical RF generator unit to the active electrode and the dispersive electrode, so as to provide RF energy from the electrosurgical RF generator unit to the active electrode; and
a control unit, which comprises circuitry and is configured to:
perform cardiac sensing for ventricular fibrillation that may be induced by the RF energy, and
upon sensing the ventricular fibrillation, apply defibrillation current to the heart.

2. The electrosurgical system according to claim 1, wherein the control unit is configured to disconnect the active electrode from the electrosurgical RF generator unit upon sensing the ventricular fibrillation.

3. The electrosurgical system according to claim 1, wherein the distal tip further comprises a defibrillation electrode, and wherein the control unit is configured to apply the defibrillation current to the heart via the defibrillation electrode.

4. The electrosurgical system according to claim 1, wherein the control unit is configured to apply the defibrillation current to the heart via the active electrode.

5. The electrosurgical system according to claim 1, wherein the distal tip further comprises a defibrillation electrode, and wherein the control unit is configured to perform the cardiac sensing using the defibrillation electrode.

6. The electrosurgical system according to claim 1, wherein the control unit is configured to perform the cardiac sensing using the active electrode.

7. The electrosurgical system according to claim 1, wherein the distal tip of the transcatheter electrosurgical device comprises a collection chamber, and wherein the distal tip further comprises insulating material disposed between the active electrode and the collection chamber.

8. The electrosurgical system according to claim 1, wherein the electrosurgical system is configured to perform transcatheter septal myectomy on the heart.

9. The electrosurgical system according to claim 8,
wherein the distal tip of the transcatheter electrosurgical device comprises a collection chamber,
wherein the active electrode is shaped as an electrically-conductive cutting ring, and
wherein the distal tip is arranged such that motion of the cutting ring, while the cutting ring contacts septal myocardial tissue of the heart and is provided with the RF energy from the RF generator unit, cuts the septal myocardial tissue and traps the cut septal myocardial tissue in the collection chamber.

10. The electrosurgical system according to claim 9,
wherein the collection chamber has an open proximal end and a closed distal end,
wherein the cutting ring is coupled to the open proximal end of the collection chamber,
wherein the transcatheter electrosurgical device further comprises an elongate cutting-ring puller, which is coupled to the cutting ring, and
wherein the distal tip is arranged such that pulling of the elongate cutting-ring puller in a proximal direction, while the cutting ring contacts the septal myocardial tissue and is provided with RF energy from the RF generator unit, pulls the cutting ring and the open proximal end of the collection chamber in the proximal direction, thereby cutting the septal myocardial tissue and trapping the cut septal myocardial tissue in the collection chamber.

11. An electrosurgical system for use with an electrosurgical radiofrequency (RF) generator unit, for performing percutaneous transcatheter septal myectomy on a heart, the electrosurgical system comprising:
(i) a transcatheter electrosurgical device, comprising:
(a) a catheter;
(b) a distal tip, which is disposable at a distal end of the catheter, and which comprises (1) a collection chamber having an open proximal end and a closed distal end; and (2) an electrically-conductive cutting ring, which is coupled to the open proximal end of the collection chamber; and
(c) an elongate cutting-ring puller, which is coupled to the cutting ring;
(ii) a dispersive electrode; and
(iii) electrical wiring, which is configured to electrically couple the electrosurgical RF generator unit to the cutting ring and the dispersive electrode, and so as to provide RF energy from the electrosurgical RF generator unit to the cutting ring,
wherein the distal tip is arranged such that pulling of the elongate cutting-ring puller in a proximal direction, while the cutting ring contacts septal myocardial tissue of the heart and is provided with the RF energy from the RF generator unit, pulls the cutting ring and the open proximal end of the collection chamber in the proximal direction, thereby cutting the septal myocardial tissue and trapping the cut septal myocardial tissue in the collection chamber.

12. The electrosurgical system according to claim 11,
wherein the collection chamber is elongatable from a short state to an elongated state, and
wherein the distal tip is arranged such that the pulling of the elongate cutting-ring puller in the proximal direction pulls the cutting ring and the open proximal end of the collection chamber in the proximal direction, thereby cutting the septal myocardial tissue, elongating the collection chamber in the proximal direction, and trapping the cut septal myocardial tissue in the collection chamber as the collection chamber elongates.

13. The electrosurgical system according to claim 12, wherein the collection chamber is accordion-pleated.

14. The electrosurgical system according to claim 11,
wherein the elongate cutting-ring puller is shaped so as to define a puller lumen, and
wherein the transcatheter electrosurgical device further comprises an elongate guide, which is slidably disposed partially within the puller lumen, and which is arranged to guide the elongate cutting-ring puller in the proximal direction during the pulling of the elongate cutting-ring puller.

15. The electrosurgical system according to claim 14, for use with a guidewire, wherein the elongate guide is shaped so as to define a guide lumen, which has a distal end opening distal to the closed distal end of the collection chamber, so as to allow passage of the guidewire distally beyond the collection chamber.

16. The electrosurgical system according to claim 15, wherein the transcatheter electrosurgical device further comprises a distal atraumatic tip, which is (a) disposed distal to the collection chamber, (b) coupled to a distal portion of the elongate guide, and (c) shaped so as to allow passage of the guidewire from the guide lumen to distally beyond the tip.

17. The electrosurgical system according to claim 11,
wherein the distal tip further comprises an atraumatic valve protector, which is disposed proximal to the cutting ring, and
wherein the distal tip is arranged such that pulling of the elongate cutting-ring puller in the proximal direction pulls the cutting ring toward the atraumatic valve protector.

18. The electrosurgical system according to claim 17, wherein the atraumatic valve protector is shaped so as to define an opening, and wherein the elongate cutting-ring puller is slidably disposed passing through the opening.
